(19) United States
(12) Patent Application Publication
(10) Pub. No.: US 2026/0066105 A1
(43) Pub. Date: Mar. 5, 2026

(54) CLINICAL DEVICE TRACKING AND MONITORING SYSTEM

(71) Applicant: Medtronic Vascular, Inc., Santa Rosa, CA (US)

(72) Inventors: James Delahunty, Galway (IE); Brian J. Kelly, Oranmore (IE); Jeffrey M. Zalewski, Corte Madera, CA (US)

(21) Appl. No.: 19/104,595

(22) PCT Filed: Apr. 7, 2023
(86) PCT No.: PCT/US2023/017914

§ 371 Date: Feb. 18, 2025

Related U.S. Application Data
(60) Provisional application No. 63/375,761, filed on Sep. 15, 2022.

(51) Int. Cl. G16H 40/63; G16H 15/00; G16H 30/40

(52) U.S. Cl. CPC G16H 40/63; G16H 15/00; G16H 30/40

(57) ABSTRACT

In one example, a medical system includes a memory; and processing circuitry communicatively coupled to the memory, the processing circuitry being configured to: obtain, during performance of a procedure in a catheterization lab on a patient, data representing an operating parameter of a medical component, the medical component comprising a medical device or a pharmacological agent; and generate, based on the data representing the operating parameter, procedure records of the procedure.

This application claims the benefit of U.S. Provisional Patent Application No. 63/375,761, filed 15 Sep. 2022, the entire contents of which is incorporated herein by reference.

### TECHNICAL FIELD

This disclosure relates to tracking and/or monitoring during a medical procedure.

### BACKGROUND

During a medical procedure, a clinician may use one or more imaging systems to be able to visualize internal anatomy of a patient. Such imaging systems may display anatomy, medical instruments, or the like, and may be used to diagnose a patient condition or assist in guiding a clinician in moving a device, such as a medical instrument to an intended location inside the patient. Imaging systems may use sensors to capture video images or still images which may be displayed during the medical procedure. Imaging systems include angiography systems, ultrasound imaging systems, computed tomography (CT) scan systems, magnetic resonance imaging (MRI) systems, isocentric C-arm fluoroscopic systems, positron emission tomography (PET) systems, intravascular ultrasound (IVUS), optical coherence tomography (OCT), near infrared spectroscopy (NIRS), as well as other imaging systems.

### SUMMARY

In general, this disclosure is directed to a clinical device tracking and monitoring system for use during a medical procedure. For example, a system may perform comprehensive automated monitoring to track an operating parameter of a medical component, such as a medical device or a pharmacological agent. The operating parameter may be any aspect associated with the medical component, such as a location of the medical component, a pressure of the medical component, a rotational speed of the medical component, power delivery parameters of the medical component, a size of the medical component, and a serial number of the medical component.

The system may include a wide variety of input modalities to facilitate tracking of the operating parameter. As one example, the system may include a wireless monitoring pad (e.g., attached to a table on which a patient is placed during a procedure). As another example, the system may include one or more cameras that generate video data of the procedure and processing circuitry configured to process the video data to determine the operating parameter. As another example, the system may include one or more microphones that generate audio data of the procedure and processing circuitry configured to process the audio data to determine the operating parameter. As another example, the system may include a medical component storage device configured to generate data representing the operating parameter (e.g., “smart storage” that communicates with the system to indicate an operating parameter of a medical component removed from the storage device). The system may include a receiving hub configured to interface with the input modalities and obtain the operating parameter.

The system may utilize the data generated via tracking/monitoring to improve one or more aspects of medical procedure performance. As one example, the system may automatically generate procedure records (e.g., based on the tracked operating parameter). As another example, the system may perform automatic inventory management. As another example, the system may control one or more operations of the procedure based on the operating parameter (e.g., adjust an indeflator driving a balloon being used in the procedure).

The system may include one or more artificial intelligence algorithms, machine learning algorithms, computer vision algorithms, or the like which the system may utilize when obtaining the operating parameter, performing tracking, or the like. For instance, the system may execute a computer vision algorithm to process video data to obtain the operating parameter.

In one example, a medical system includes memory and processing circuitry communicatively coupled to the memory, the processing circuitry being configured to: obtain, during performance of a procedure in a medical facility on a patient, data representing an operating parameter of a medical component, the medical component comprising a medical device or a pharmacological agent; and generate, based on the data representing the operating parameter, procedure records of the procedure.

In another example, a method includes obtaining, during performance of a procedure in a medical facility on a patient, data representing an operating parameter of a medical component, the medical component comprising a medical device or a pharmacological agent; and generating, based on the data representing the operating parameter, procedure records of the procedure.

In another example, a non-transitory computer readable medium stores instructions, which, when executed, cause processing circuitry to obtain, during performance of a procedure in a medical facility on a patient, data representing an operating parameter of a medical component, the medical component comprising a medical device or a pharmacological agent; and generate, based on the data representing the operating parameter, procedure records of the procedure.

In another example, a medical system includes memory; and processing circuitry communicatively coupled to the memory, the processing circuitry being configured to: obtain, during performance of a procedure in a medical facility on a patient, data representing a radiation exposure of a person in the medical facility; and generate, based on the data representing the radiation exposure, an exposure report for the person.

In another example, a method includes obtaining, during performance of a procedure in a medical facility on a patient, data representing a radiation exposure of a person in the medical facility; and generating, based on the data representing the radiation exposure, an exposure report for the person.

In another example, a non-transitory computer-readable storage medium stores instructions, which, when executed, cause processing circuitry to: obtain, during performance of a procedure in a medical facility on a patient, data representing a radiation exposure of a person in the medical facility; and generate, based on the data representing the radiation exposure, an exposure report for the person.

These and other aspects of the present disclosure will be apparent from the detailed description below. In no event, however, should the above summaries be construed as limitations on the claimed subject matter, which subject matter is defined solely by the attached claims.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIG. 1 is a schematic perspective view of one example of a system for performing tracking in a Cath lab, in accordance with one or more aspects of this disclosure.

FIG. 2 is a block diagram of one example of a computing device, in accordance with one or more aspects of this disclosure.

FIG. 3 is a conceptual diagram illustrating an example medical component having an operating parameter configured to be tracked by a system, in accordance with one or more aspects of this disclosure.

FIG. 4 is a schematic perspective view of one example of a system for performing radiation tracking in a Cath lab, in accordance with one or more aspects of this disclosure.

FIG. 5 is a flow diagram illustrating example techniques for tracking and/or monitoring in a Cath lab, in accordance with one or more aspects of the present disclosure.

FIG. 6 is a flow diagram illustrating example techniques for tracking and/or monitoring radiation in a Cath lab, in accordance with one or more aspects of the present disclosure.

### DETAILED DESCRIPTION

In general, this disclosure is directed to a clinical device tracking and monitoring system for use during such a medical procedure. During a medical procedure, such as a procedure performed in a catheterization laboratory (or “Cath lab”) for interventional cardiology, many medical components may be utilized. Records of medical component use during a procedure may be manually performed (e.g., by a charting nurse or other clinician). However, even when performed accurately, such manual recordation may not capture a full picture of which medical components were used and/or how medical components were used.

In accordance with one or more aspects of this disclosure, a system may perform comprehensive automated medical device real-time tracking and monitoring. The techniques of this disclosure may be applied in a catheterization laboratory (or “Cath lab”) for interventional cardiology, though could be extended further to operating theatres and even for general use in various other clinical settings (e.g., in medical facilities). Example procedures include, but are not limited to, coronary procedures (angioplasty, stenting, diagnostic catheterization, rotational or laser atherectomy, IVL), denervation procedures (e.g., renal denervation or hepatic denervation or other denervation using electrical, chemical, ultrasonic, or other energy), and structural heart procedures (e.g., catheter-based valve repair or replacement).

In some examples, the system may include one or more clinical monitoring cameras. The clinical monitoring camera may be a camera in the Cath lab with a view of people (e.g., patient and/or clinicians (e.g., physician(s), nurses, and other personnel)) in the room. The clinical monitoring camera may be a camera implemented specifically for this purpose or may be a pre-existing camera in the room which is adopted for this purpose. The system may utilize computer vision to track and interpret clinical workflows and/or identify & track medical devices and pharmacological agents in the Cath lab (e.g., based on video data generated by the clinical monitoring camera).

In some examples, the system may include one or more microphones. The microphones may be separate modules, or may be physically integrated into the camera module. The system can use natural language processing to parse clinical proceedings, notes, and verbal discussion (e.g., based on audio data generated by the microphones).

The system may track, monitor, and/or identify medical devices and/or medications (e.g., obtain an operating parameter of a medical component). The system may obtain the operating parameter by performing direct tracking & identification of the medical component themselves, and/or monitoring physical measurements using accessory attachment devices. The system may perform the tracking, monitoring, and/or identifying via wireless transmitters using RFID, NFC, RF, Bluetooth and/or others. These transmitters may be passive or active and may include a battery to provide power for sensing & transmitting. The transmitters may be attached to the medical components. The transmitters may also include conductive or inductive charging components. In some examples, some devices may involve a wired system (e.g., wired transmitters with connectors to link sensor devices to the device monitoring system). In some examples, one or more of the transmitters may include sensors configured to read physical measurements (e.g., angioplasty balloon pressure).

The system may include a receiving hub, which may receive the signals transmitted by the transmitters. The receiving hub may be a pad placed directly on top of or below the patient table, or a surface placed near the patient table, within or beyond the sterile field. The receiving hub may also include a conductive or inductive charging system for powering medical device sensor systems and batteries. Multiple hubs may be implemented throughout the room (e.g., the Cath lab), allowing for wireless triangulation to locate devices. In some examples, the receiving hub may include of electronic connection ports, allowing wired devices to be plugged in to the receiving hub.

In some examples, the system may include an inventory tracking system. The inventory tracking system may include a barcode scanner (or multiple scanners), a vision system, or a wireless signal receiver (e.g., which would identify medical devices and pharmacological agents in storage and/or entering/leaving storage). The data generated by the inventory tracking system may supplement other device tracking & identification elements in this system and may allow for exact product identification in addition to lot numbers and other such information. This inventory tracking system could be implemented within storage cabinets, shelves, or at a location the medical component would pass through on the medical component's journey into or out of storage and/or the sterile field.

In some examples, the system may obtain the operating parameter via screen capture from other displays (e.g., other displays in the Cath lab). For instance, processing circuitry of the system may receive data representing video data displayed at the other displays (e.g., via direct wire connection or cameras pointed at the other displays), and process said data to obtain the operating parameter.

The system may use computational algorithms from these combined elements to identify devices & medications, track their locations & workflow status, capture measurements, analyze, use for artificial intelligence (A1) inference in other medical systems, and collect data to train A1 models. This can be used to directly present real-time information to hospital personnel such as inventory tracking, warnings, guidance & informatics.

The system may monitor and interprets Cath lab proceedings in real-time and can produce an alert when a potential miscommunication is detected (e.g., a physician asks for 3 mm balloon, but the visual system detects that they've been handed a 4 mm balloon). The alert may consist of an audible notification from our system and/or via a visual warning displayed on a screen. This visual warning may take the form of a graphical/text-based warning superimposed on a screen being used to display other clinical information.

The system may perform anonymization. For instance, the system may perform automatic face blurring, blurring of personally-identifying text, and utilize alphanumerical codes to identify people when deemed appropriate. The system may allow users to edit and select elements of the procedure to be recorded or redacted through an interface with software tools to facilitate this and A1 models dedicated to automating suggested inclusions and redactions.

The system may enable automatic generation of procedure records. Video & audio is not necessarily included in the procedure records, but may be transcribed and selectively included. The user can also choose to upload selected data and imagery to other information systems.

Radiation may be emitted during Cath lab procedures. In general, it may be desirable to track how much radiation people, such as clinicians, are exposed. Such tracking may be performed by each clinician carrying a dosimeter. However, carrying dosimeters may present one or more disadvantages. As one example, a clinician may forget to carry a dosimeter during one or more procedures. As another example, dosimeters merely provide a single measurement, where certain body parts of a clinician may experience higher doses than indicated by a dosimeter worn by the clinician. As another example, the use of worn dosimeters may not be conducive to tracking radiation dosages across multiple procedures.

In accordance with one or more aspects of this disclosure, a system may perform radiation exposure tracking and/or mapping. For instance, the system may, in addition to or in place of carried dosimeters, track radiation exposure of persons in a Cath lab. As one example, the system may utilize video data from one or more cameras in the Cath lab, and process the video data to determine radiation exposure dose for a clinician. For instance, the system may process the video data to determine a location of the clinician within the Cath lab, and determine the radiation exposure dose based on a comparison of the location of the clinician with a location of a radiation emitting device.

The system may perform one or more actions based on the radiation exposure tracking/mapping. As one example, the system may generate an exposure report (e.g., a report that indicates a radiation dose experienced by a clinician). As another example, the system may separately track radiation exposure doses of multiple body parts of a clinician. As another example, the system may provide recommendations (e.g., stand in a different location, wear more or less radiation protection equipment, etc.).

As noted above, aspects of this disclosure are applicable to at least Cath lab procedures. Example Cath lab procedures include, but are not necessarily limited to, coronary procedures, renal denervation (RDN) procedures, structural heart and aortic (SH&A) procedures (e.g., transcatheter aortic valve replacement (TAVR), transcatheter mitral valve replacement (TMVR), and the like), device implantation procedures (e.g., heart monitors, pacemakers, defibrillators, and the like).

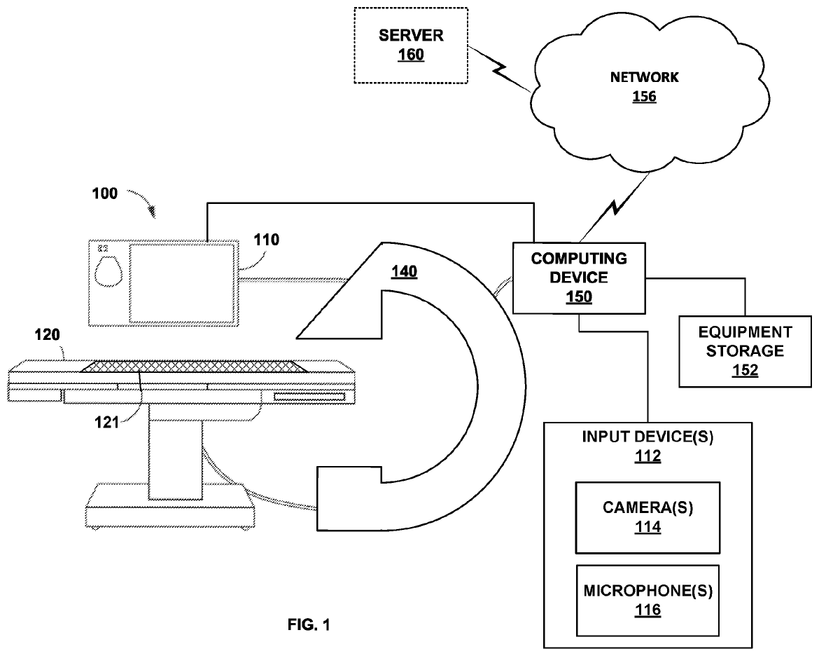

FIG. 1 is a schematic perspective view of one example of a system for performing tracking in a Cath lab, in accordance with one or more aspects of this disclosure. Medical system 100 may constitute a system for tracking an operating parameter of a medical component and/or tracking radiation exposure of clinicians. Such a system may facilitate identification and/or record keeping for medical components.

System 100 includes a display device 110, a table 120, device tracking system 121, imager 140 (which may be an angiography and/or fluoroscopy imager), additional imager(s) 142, computing device 150, input device(s) 112, equipment storage 152, server 160, and network 156. System 100 may be an example of a system for use in a Cath lab. In some examples, system 100 may include other devices. In some examples, system 100 may be used during a diagnostic session to diagnose cardiovascular issues for a patient. In some examples, system 100 may be used during a medical procedure (e.g., an intervention to treat a cardiovascular issue, such as a lesion).

Computing device 150 may be associated with one or more clinicians, who may be located in the Cath lab during the medical procedure. Computing device 150 may include, for example, an off-the-shelf device, such as a laptop computer, desktop computer, tablet computer, smart phone, or other similar device. In other examples, computing device 150 may be a special purpose computing device, such as one specifically designed to be used in a Cath lab. Computing device 150 includes memory and processing circuitry.

Computing device 150 may be configured to control an indeflator, an electrosurgical generator, a peristaltic pump, a power supply, or any other accessories and peripheral devices relating to, or forming part of, system 100. In some examples, computing device 150 may perform various control functions with respect to imager 140, display device 110, input devices 112, equipment storage 152, and/or the like. Computing device 150 may be communicatively coupled to device tracking system 121, imager 140, input devices 112, equipment storage 152, display device 110, server 160, and/or network 156.

While a number of features are described herein as being attributed to computing device 150, in some examples, features attributed to computing device 150 may be performed by processing circuitry of any of computing device 150, imager 140, server 160, network 156 (e.g., one or more computing devices forming or connected to network 156), other elements of system 100, or any combinations thereof. In some examples, processing circuitry associated with computing device 150 may be distributed and shared across any combination of computing device 150, input devices 112, equipment storage 152, imager 140, server 160, network 156, display device 110, and/or other elements of system 100. Additionally, in some examples, processing operations or other operations performed by processing circuitry of computing device 150 may be performed by processing circuitry residing remotely, such as one or more cloud servers or processors. For purposes of ease of discussion herein, such processing circuitry may be considered a part of computing device 150.

System 100 may include network 156, which is a suitable network such as a local area network (LAN) that includes a wired network or a wireless network, a wide area network (WAN), a wireless mobile network, a Bluetooth network, or the Internet. In some examples, network 156 may be a secure network, such as a hospital network, which may limit access by users. In some examples, network 156 may interconnect various devices of system 100.

As discussed above, imager 140 may be an angiography and/or fluoroscopy imager, and may image portions of a patient's body during or before a Medical procedure to visualize characteristics and locations of lesions inside, for example a cardiac vasculature of the patient.

Input devices 112 may represent component configured to receive and/or generate data. As shown in FIG. 1, input devices 112 may include cameras 114 and microphones 116. However, in other examples, input devices 112 may include more or fewer components.

Cameras 114 may be configured to generate video data representative of scenes in the Cath Lab. Computing device 150 may be configured to receive the video data during the medical procedure.

Microphones 116 may be configured to generate audio data representative of audio in the Cath lab. Computing device 150 may be configured to receive audio data from microphones 116 during the medical procedure, as is discussed later in this disclosure. Microphones 116 may be off the shelf components of computing device 150, a laptop, tablet, mobile phone, or the like or may be a part of a Cath Lab. Microphones 116 may be stand-alone or may be integrated into cameras 114.

Computing device 150 may be configured to execute one or more artificial intelligence (AI), machine learning (ML), and/or computer vision algorithms to process video data (e.g., video data generated by cameras 114). For instance, computing device 150 may process the video data to perform tracking of medical components and/or clinicians during a procedure. As one example, computing device 150 may process the video data to recognize packaging of medical components, QR codes associated with medical components, bar codes associated with medical components, or the like. As another example, computing device 150 executing the one or more computer vision algorithm(s) may determine the devices used and update an inventory of such devices (e.g., deduct the devices from a stored inventory log).

Computing device 150 may be configured to execute one or more natural language processing algorithms to discern between clinically relevant and non-clinically relevant spoken words or phrases which may be captured during a medical procedure by, for example, one or more microphones 116.

Additional equipment 152 may include devices configured to be used during a medical procedure, such as a PCI procedure, including, but not limited to, stents, catheters, angioplasty devices, ablation devices, atherectomy devices, energy generation devices, smart manifolds, device add-ons, or other such devices.

Display device 110 may be configured to display captured imaging data, from, for example, imager 140. In some examples, display device 110 may be configured to display a 3D model of the coronary vasculature of a patient. In some examples, display device 110 may be configured to display the various user interfaces disclosed herein. In some examples display device 110 may be configured to display procedural guidance as disclosed herein and/or information overlaid onto angiogram imagining data. Display device 110 may be configured to display any other content discussed as being displayed in this disclosure.

In some examples, computing device 150 may receive a representation of what is being displayed at display device 110. As one example, computing device 150 may be connected to, or connected in-line with, display device 110. As another example, computing device 150 may receive a video signal from a camera (e.g., a camera of cameras 114) that is directed at display device 110.

Table 120 may be, for example, an operating table or other table suitable for use during a medical procedure, such as a PCI procedure. Table 120 may include a device tracking system 121, such as a specially designed pad to be placed under, or integrated into, Table 120. Device tracking system 121 may, in some examples, be placed on top of the patient or integrated into sterile drapes placed on top of the patient. In some examples, device tracking system 121 may be placed on a sterile prep table. For instance, an additional device tracking system 121 may be placed on the sterile prep table to facilitate more detailed tracking of devices or have different capabilities to a version of device tracking system 121 on the Cath lab table.

In some examples, one or more components of device tracking system 121 may be disposable. For instance, as discussed above, one or more components of device tracking system 121 may be integrated into sterile drapes. In some examples, one or more components of device tracking system 121 may be reusable. For instance, a version (e.g., a more feature rich version) of device tracking system 121 may be placed on the prep table under sterile drapes. As another example, one or more components of device tracking system 121 may be integrated into the prep table (e.g., a smart sterile prep table).

Device tracking system 121 may include radio frequency identification (RFID), near field communication (NFC), battery powered sensors, triangulation technology, and/or an electromagnetic (EM) field generator which may be used to generate an EM field during the medical procedure. Such technologies may be used to track the positions of one or more devices within the body of a patient during a medical procedure. For example, device tracking system may track the location of devices (e.g., devices of additional equipment 152) by tracking sensors attached to or incorporated in such devices. In some examples, device tracking system 121 may serve as a charging pad which may wirelessly charge various sensors which may be placed on or in the patient, such as for monitoring patient parameters, during the medical procedure. Such sensors may wirelessly communicate with computing device 150. In this manner, fewer wires may be present in a Cath lab than otherwise may be, lowering a risk of entanglement with the patient or a clinician moving about the Cath lab.

Equipment storage system 152 may be configured to store and/or provide medical components (e.g., for use in a Cath lab procedure). For instance, equipment storage system 152 may be a so called “smart storage” device that outputs an indication (e.g., to computing device 150) in response to a medical component being removed from equipment storage system 152. As one example, equipment storage system 152 may include an RFID scanner that scans RFID tags of medical components (e.g., as the medical components are removed from equipment storage system 152).

Server 160 may be configured to store data obtained by and/or determined or generated by computing device 150. In some examples, server 160 may be configured to perform techniques attributed to computing device 150. Server 160 may be communicatively coupled to computing device 150, for example, by wired, optical, or wireless communications and/or by network 156. Server 1060 may be a hospital server which may or may not be located in a Cath lab, such as a cloud-based server, or the like. Server 1060 may be configured to store patient data, electronic patient records, or the like.

In some examples, system 100 may include an automated contrast delivery device. In such examples, system 100 may monitor an amount of contrast provided to the patient by the automated contrast delivery device or otherwise provided to the patient. Computing device 150, based on the amount of contrast provided to the patient and a first amount of contrast needed or recommended for obtaining further desired imaging data, control the automated contrast delivery device to deliver a second amount of contrast.

In accordance with one or more aspects of this disclosure, system 100 may perform comprehensive automated monitoring to track an operating parameter of a medical component, such as a medical device or a pharmacological agent. For instance, computing device 150 may obtain, during performance of procedure in a catheterization lab on a patient, data representing an operating parameter of a medical component. As one example, computing device 150 may receive, via device tracking system 121, a location of a medical device. As another example, computing device 150 may receive, via device tracking system 121, a pressure of a balloon used in performance of the procedure (e.g., via an in-line pressure sensor). As another example, computing device 150 may receive, via a camera of cameras 114, video data and process the video data to determine the location of the medical device. Examples of medical devices include, but are not necessarily limited to, sensors, catheters, balloons, rotational devices, laser atherectomy devices, Intravascular Lithotripsy (IVL) devices, renal denervation devices, and the like.

System 100 may perform one or more operations based on the tracking of the operating parameter. As one example, computing device 150 may generate, based on the data representing the operating parameter, procedure records of the procedure. The procedure records may include the operating parameter. As one example, where the operating parameter is a location of a medical device, the procedure records may include a time series of locations of the medical device (e.g., a series of x,y,z or other coordinates). As another example, where the operating parameter is a pressure of the medical component, the procedure records may include a time series of pressure values (e.g., of a balloon).

As noted above, system 100 may perform automated inventory tracking. Tracking and recording devices used during procedures may undesirably add time and burden to staff in clinical settings. Additionally, tracking devices is often performed on paper (e.g., does not link directly to a centralized inventory management system). As such, inventory management is often performed either via a manual stock-checking process or via rough estimations of typical product usage. These approaches are burdensome and inefficient.

In accordance with one or more aspects of this disclosure, system 100 may perform tracking (e.g., automated and/or passive) of medical components entering or exiting storage (e.g., equipment storage system 152) and/or the sterile field. As noted above, medical components may include medical devices, accessories, and/or pharmacological agents. As also noted above, system 100 may perform automated tracking of when & how each medical component is used. System 100 may link what device settings were applied with each device (e.g., pressure applied to balloon). Settings recorded via a sensor datalogger or via visual means (e.g., cameras 114 and computing device 150 visually tracking balloon expansion). System 100 may passively track quantities of pharmacological agents given to the patient (e.g., via natural language processing, visual means, or via sensors attached to injection devices). Such tracking may be considered an example of obtaining operating parameters of medical components.

In some examples, medical components may include (e.g., carry, have attached thereon, etc.) unique device identifiers. Such identifiers may include RFID tags and/or barcodes. System 100 may scan medical components when said medical components leave a storage cabinet/area and/or into a sterile field. The scanner may be triggered when the product passes through a window or when it moves away from/towards a surface (e.g., a rear wall of equipment storage system 152). Additionally or alternatively, system 100 may track the medical component via visual means.

Alternatively, the product status/location may be tracked by visual means. For instance, computing device 150 may receive video data from camera 114 and execute a computer vision algorithm (e.g., a machine learning algorithm trained to recognize medical device packaging and products) to track the medical component. This algorithm may also be trained to identify the specific product model. Such tracking may be considered an example of obtaining operating parameters of medical components.

System 100 may be configured to output an indication of an inventory list. For instance, computing device 150 may cause display 110 (or another display) to output a live/recent inventory list on a screen in the Cath lab, operating room, or any other clinical room. In some examples, computing device 150 may send this information to a centralized database. In some examples, computing device 150 may reorder new inventory through this system in an automated manner or via a streamlined interface.

The generation of procedure records and/or automated tracking may provide one or more advantages. As one example, by automatically generating procedure records and/or automated tracking, computing device 150 may reduce administrative and/or reporting burden (e.g., no need for nurses to physically record products used, and/or automated/streamlined inventory management). As another example, by automatically generating procedure records and/or automated tracking, computing device 150 may improve procedure workflows (e.g., no need for nurses to actively record settings used per device or quantities given to patient). As another example, by automatically generating procedure records and/or automated tracking, computing device 150 may improve data management (e.g., digital records automatically created, and/or simplifies device identification process, such as for training video purposes).

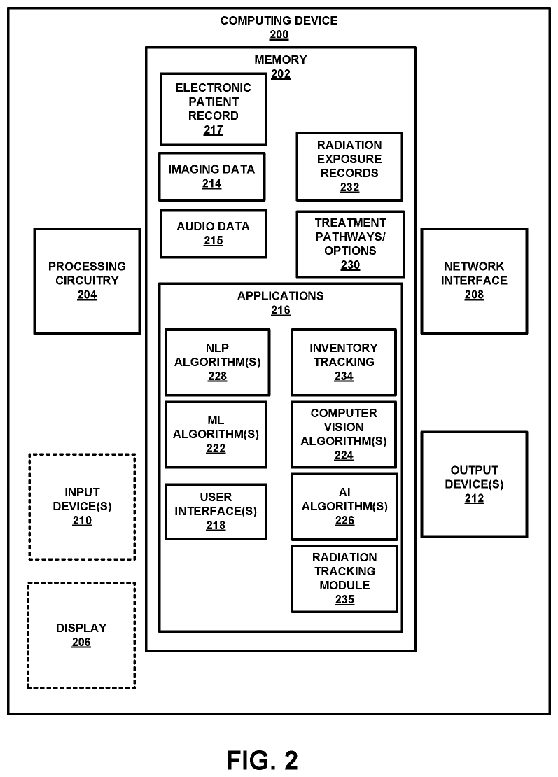

FIG. 2 is a block diagram of one example of a computing device, in accordance with one or more aspects of this disclosure. Computing device 200 may be an example of computing device 150, a computing device of network 156, and/or server 160 of FIG. 1 and may include a workstation, a desktop computer, a laptop computer, a server, a smart phone, a tablet, a dedicated computing device, or any other computing device capable of performing the techniques of this disclosure.

In some examples, computing device 200 may be configured to perform processing, control and other functions associated with various devices of FIG. 1, such as display device 110, input devices 112, imager 140, equipment storage 152, and/or device tracking system 121. Computing device 200 may include, for example, a memory 202, processing circuitry 204, a display 206, a network interface 208, an input device(s) 210, or an output device(s) 212, each of which may represent any of multiple instances of such a device within the computing system, for ease of description.

While processing circuitry 204 appears in computing device 200 in FIG. 2, in some examples, features attributed to processing circuitry 204 may be performed by processing circuitry of any of computing device 150, imager 140, server 160, computing devices of network 156, or other components of FIG. 1. In some examples, one or more processors associated with processing circuitry 204 in computing device 200 may be distributed and shared across any combination of computing device 150, imager 140, server 160, computing devices of network 156, or other components of FIG. 1. Additionally, in some examples, processing operations or other operations performed by processing circuitry 204 may be performed by one or more processors residing remotely, such as one or more cloud servers or processors, each of which may be considered a part of computing device 200. Computing device 200 may be used to perform any of the techniques described in this disclosure, and may form all or part of devices or systems configured to perform such techniques, alone or in conjunction with other components, such as components of computing device 150, imager 140, server 160, computing devices of network 156, other components of FIG. 1, or a system including any or all of such devices.

Memory 202 of computing device 200 includes any non-transitory computer-readable storage media for storing data or software that is executable by processing circuitry 204 and that controls the operation of computing device 150. In one or more examples, memory 202 may include one or more solid-state storage devices such as flash memory chips. In one or more examples, memory 202 may include one or more mass storage devices connected to the processing circuitry 204 through a mass storage controller (not shown) and a communications bus (not shown).

Although the description of computer-readable media herein refers to a solid-state storage, it should be appreciated by those skilled in the art that computer-readable storage media may be any available media that may be accessed by the processing circuitry 204. That is, computer readable storage media includes non-transitory, volatile and non-volatile, removable and non-removable media implemented in any method or technology for storage of information such as computer-readable instructions, data structures, program modules, or other data. For example, computer-readable storage media includes RAM, ROM, EPROM, EEPROM, flash memory or other solid state memory technology, CD-ROM, DVD, Blu-Ray or other optical storage, magnetic cassettes, magnetic tape, magnetic disk storage or other magnetic storage devices, or any other medium that may be used to store the desired information and that may be accessed by computing device 200. In one or more examples, computer-readable storage media may be stored in the cloud or remote storage and accessed using any suitable technique or techniques through at least one of a wired or wireless connection.

Memory 202 may store NLP algorithm(s) 228, ML algorithm(s) 222, AI algorithm(s) 226, computer vision algorithm(s) 224, inventory tracking algorithm(s) 234, radiation tracking module 235, and/or user interface(s) 218. In some examples, any of ML algorithm(s) 222, AI algorithm(s) 226, computer vision algorithm(s) 224, and/or NLP algorithm(s) 228 may be the same. In some examples, any of ML algorithm(s) 222, AI algorithm(s) 226, computer vision algorithm(s) 224, and/or NLP algorithm(s) 228 may be the different.

Memory 202 may also store user interface(s) 218 and/or inventory tracking algorithm(s) 234. User interface(s) 218 may include one or more user interfaces which processing circuitry 204 may output for display by display 206 and/or display device 110. Inventory tracking algorithm(s) 234 may be used to track inventory of devices used during a medical procedure. For example, a clinician may scan a QR code or a bar code of a device using input device(s) 210 and processing circuitry 204 executing inventory tracking algorithm(s) 234 may update inventory of such devices. In some examples, processing circuitry 204 may execute computer vision algorithm(s) 236 to determine which devices are being used during the procedure and update inventory tracking algorithm(s) 234 (or an inventory otherwise in memory 202) to track inventory, for example, of additional equipment 152.

Memory 202 may store imaging data 214, audio data 215, electronic patient record 217, and/or radiation exposure records 232. Imaging data 214 may be captured by imager 140 and/or cameras 114 (FIG. 1) during a medical procedure of a patient. Processing circuitry 204 may obtain imaging data 214 from imager 140 and/or cameras 114 and store imaging data 214 in memory 202. Processing circuitry 204 may use imaging data 214 to determine 3D model and/or update radiation exposure records 232. Audio data 215 may be captured by microphones 116 (FIG. 1) during a medical procedure of a patient. Processing circuitry 204 may use information obtained during a medical procedure to automatically update electronic patient record 217 such that a clinician does not need to enter all pertinent information into electronic patient record 217 manually.

Any or all of ML algorithm(s) 222, computer vision algorithm 224, and/or AI algorithm(s) 226, may be trained using data collected from past medical procedures, such as imaging data, device data (e.g., including device parameters such as device size, length, device settings, etc.) or the like. Device settings may include time used, pressure used, or the like. For example, ML algorithm(s) 222, computer vision algorithm 224, and/or AI algorithm(s) 226, may be trained on data from actual procedures, reflecting actual treatments and actual outcomes from past medical procedures. Such algorithms may be utilized to determine 3D model 232, clinical guidance 220, and/or treatment pathways/options 230.

For example, ML algorithm(s) 222 may include a k-means clustering model which may have a plurality of clusters: one for each particular treatment technique (e.g., treatment pathway or treatment option) using one or more particular devices. Each identified lesion may be associated with a vector that includes variables for, e.g., type of coronary issue, severity of the coronary issue, complexity of the coronary issue, location of the coronary issue, classification of a lesion, anatomy in the area of the coronary issue, other anatomy, comorbidities of the patient, cholesterol level, blood pressure, blood oxygenation, age, physical exercise level, and/or the like. The location of the vector in a given one of the clusters may be indicative of a particular treatment using one or more particular devices. For example, if the vector falls within the cluster for angioplasty using a particular device, machine learning model(s) 222 may include angioplasty as a treatment pathway and angioplasty with the particular device as a treatment option which processing circuitry 204 may store in treatment pathways/options 230.

Alternatively, the k-means clustering algorithm may have a plurality of clusters, one for each type of lesion. Each treatment strategy may be associated with a vector that includes variables for, e.g., type of coronary issue, severity of the coronary issue, complexity of the coronary issue, location of the coronary issue, anatomy in the area of the coronary issue, other anatomy, comorbidities of the patient, cholesterol level, blood pressure, blood oxygenation, age, physical exercise level, and/or the like.

Other potential machine learning or artificial intelligence techniques that may be used include Naïve Bayes, k-nearest neighbors, random forest, support vector machines, neural networks, linear regression, logistic regression, etc.

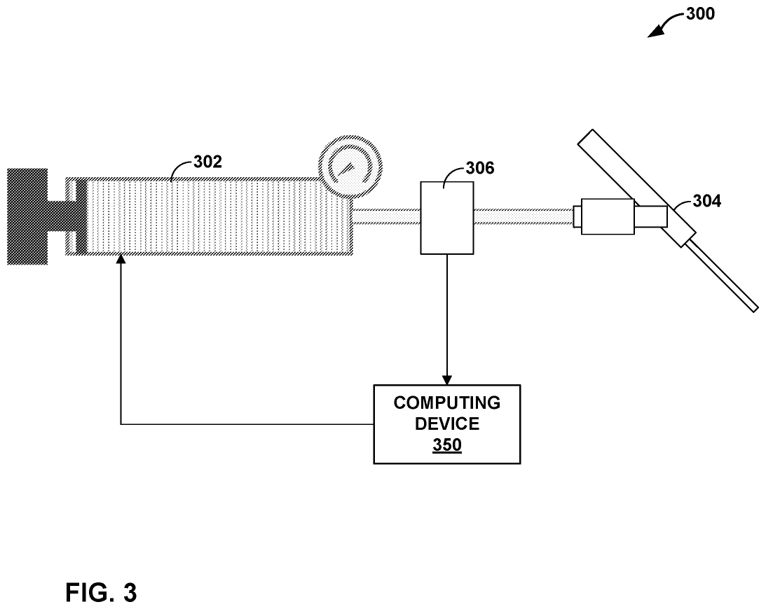

Processing circuitry 204 may execute radiation tracking module 235 to track a radiation exposure dosage of one or more clinicians. Further details of one example of radiation tracking module 235 are discussed below with reference to FIG. 3.

Processing circuitry 204 may execute any of user interface(s) 218 so as to cause display 206 (and/or display device 110 of FIG. 1) to present that UI of user interface(s) 218 to one or more clinicians performing the therapeutic medical procedure. For instance, processing circuitry 204 may execute a user interface of user interfaces 218 to cause display 206 to output a radiation tracking heatmap.

Processing circuitry 204 may be implemented by one or more processors, which may include any number of fixed-function circuits, programmable circuits, or a combination thereof. In various examples, control of any function by processing circuitry 204 may be implemented directly or in conjunction with any suitable electronic circuitry appropriate for the specified function. Fixed-function circuits refer to circuits that provide particular functionality and are preset on the operations that may be performed. Programmable circuits refer to circuits that may programmed to perform various tasks and provide flexible functionality in the operations that may be performed. For instance, programmable circuits may execute software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. In some examples, the one or more of the units may be distinct circuit blocks (fixed-function or programmable), and in some examples, the one or more units may be integrated circuits.

Instructions may be executed by one or more processors, such as one or more digital signal processors (DSPs), general purpose microprocessors, application specific integrated circuits (ASICs), field programmable gate arrays (FPGAs), graphics processing units (GPUs) or other equivalent integrated or discrete logic circuitry. Accordingly, the term processing circuitry 204 as used herein may refer to one or more processors having any of the foregoing processor or processing structure or any other structure suitable for implementation of the techniques described herein. In addition, in some aspects, the functionality described herein may be provided within dedicated hardware or software modules configured for encoding and decoding, or incorporated in a combined codec. Also, the techniques could be fully implemented in one or more circuits or logic elements.

Display 206 may be touch sensitive or voice activated, enabling display 206 to serve as both an input and output device. Alternatively, a keyboard (not shown), mouse (not shown), joystick (not shown) or other data input device(s)s (e.g., input device(s) 210) may be employed. In some examples, display 206 may include a virtual reality and/or augmented reality headset. In some examples, display 206 may include a hologram device.

Network interface 208 may be adapted to connect to a network (e.g., network 156) such as a local area network (LAN) that includes a wired network or a wireless network, a wide area network (WAN), a wireless mobile network, a Bluetooth network, or the internet. In some examples, network interface 208 may include one or more application programming interfaces (APIs) for facilitating communication with other devices. For example, computing device 200 may receive imaging data 214 from imager 140 and/or additional imager(s) 142 during a medical procedure via network interface 208. Computing device 200 may interact with server 160 via network interface 208. Computing device 200 may receive updates to its software, for example, applications 216, via network interface 208. Computing device 200 may also display notifications on display 206 that a software update is available.

Input device(s) 210 may be an example of input devices 112 of FIG. 1. Input device(s) 210 may be any device that enables a user to interact with computing device 200, such as, for example, a mouse, joystick, camera, microphone, keyboard, foot pedal, touch screen, augmented-reality input device(s) receiving inputs such as hand gestures or body movements, or voice interface.

Output device(s) 212 may include any connectivity port or bus, such as, for example, parallel ports, video ports (e.g., high-definition media interface (HDMI), DisplayPort, etc.), serial ports, universal serial busses (USB), or any other similar connectivity port known to those skilled in the art. Applications 216 may be one or more software programs stored in memory 202 and executed by processing circuitry 204 of computing device 200.

FIG. 3 is a conceptual diagram illustrating an example medical component having an operating parameter configured to be tracked by a system, in accordance with one or more aspects of this disclosure. As shown in FIG. 3, system 300 may include indeflator 302, balloon catheter 304, pressure sensor 306, and computing device 350. Computing device 350 may be an example of computing device 150 of FIG. 1. System 300 may include additional components, or may omit some components of FIG. 3. As one example, system 300 may include a sensor configured to measure an amount of medication (or other fluid) administered to the patient.

Balloon catheter 304 may be a catheter configured to be inserted into a patient and, at a target location, inflated. Balloon catheter 304 may be driven (e.g., caused to inflate or deflate) by indeflator 302. Pressure sensor 306 may be configured to sense a pressure at which balloon catheter 304 is being driven (e.g., by indeflator 302). Pressure sensor 306 may be connected (e.g., wired or wirelessly) to computing device 350. In some examples, pressure sensor 306 may be a ultrasound doppler flow meter that attaches (e.g., clips) onto indeflator tubing to (indirectly) quantify balloon pressure.

In operation, pressure sensor 306 may output data representing the pressure at which balloon catheter 304 is being driven, and said data may be received by computing device 350. This data may be an example of an operating parameter of a medical component. Computing device 350 may perform one or more actions based on the data representing the pressure at which balloon catheter 304 is being driven. As one example, computing device 350 may generate procedure records that include the pressure. As another example, computing device 350 may adjust operation of indeflator 302 based on the pressure (e.g., reduce a pressure provided by indeflator 302 responsive to the pressure being greater than a threshold pressure).

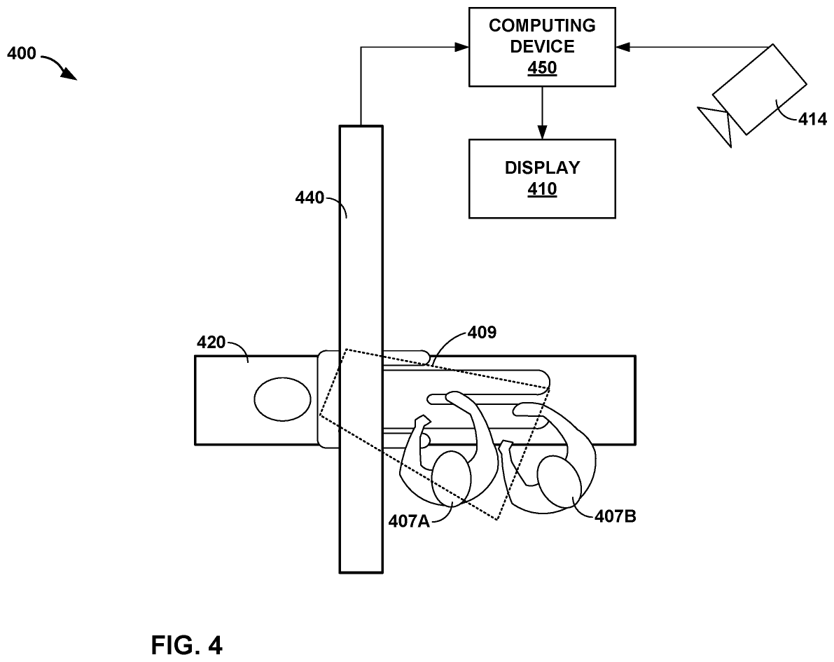

FIG. 4 is a schematic perspective view of one example of a system for performing radiation tracking in a Cath lab, in accordance with one or more aspects of this disclosure. System 400 of FIG. 4 may be considered an example of system 100 of FIG. 1. Similarly, display 410, camera 414, table 420, imager 440, and computing device 450 of FIG. 4 may be considered examples of display 110, camera 114, table 120, imager 140, and computing device 150 of FIG. 1. Clinicians 407A and 407B may be present in the Cath lab.

Radiation may be emitted during Cath lab procedures (e.g., by imager 440). In general, it may be desirable to track how much radiation people, such as clinicians 407A and 407B, are exposed. Such tracking may be performed by each clinician carrying a dosimeter. However, carrying dosimeters may present one or more disadvantages. As one example, a clinician may forget to carry a dosimeter during one or more procedures. As another example, dosimeters merely provide a single measurement, where certain body parts of a clinician may experience higher doses than indicated by a dosimeter worn by the clinician. As another example, the use of worn dosimeters may not be conducive to tracking radiation dosages across multiple procedures.

In accordance with one or more aspects of this disclosure, system 400 may perform radiation exposure tracking and/or mapping. For instance, system 400 may, in addition to or in place of carried dosimeters, track radiation exposure of clinicians 407A and/or 407B in a Cath lab. As one example, system 400 may utilize video data from one or more cameras in the Cath lab (e.g., camera 414), and process the video data to determine radiation exposure dose for clinicians 407A and/or 407B. For instance, system 400 may process the video data to determine a location of clinicians 407A and/or 407B within the Cath lab, and determine the radiation exposure dose based on a comparison of the location of clinicians 407A and/or 407B with a location of a radiation emitting device (e.g., imager 140).

In some examples, to determine the radiation exposure dose based on a comparison of the location of clinicians 407A and/or 407B with a location of a radiation emitting device, computing device 450 may determine one or more parameters of the radiation emitting device (e.g., one or more of a direction of emission, a focus of emission, and an intensity of emission). The determined parameters may define radiation in emission field 409 (e.g., generated by imager 440). Computing device 450 may determine a position of clinicians 407A and/or 407B relative to the radiation emission device (e.g., relative to emission field 409). Based on the parameters and the position of clinicians 407A and/or 407B, computing device 450 may determine radiation exposure doses of clinicians 407A and/or 407B. In some examples, computing device 450 may factor in other parameters when determining the radiation exposure doses (e.g., blocking equipment, other large masses, surfaces, walls, etc).

In some examples, system 400 may determine a single radiation exposure dose for one or both of clinicians 407A and/or 407B. In other examples, system 400 may determine multiple single radiation exposure doses for one or both of clinicians 407A and/or 407B. For instance, system 400 may determine a respective radiation dose for multiple body parts of clinicians 407A and/or 407B. As one example, system 400 may determine a first radiation dose for a first hand of clinician 407A, a second radiation dose for a second hand of clinician 407A, a third radiation dose for a head of clinician 407A, etc. When determining such multiple doses, system 400 may similarly track positions of the body parts (e.g., relative to the radiation emission device).

In some examples, system 400 may perform calibrate the image based radiation tracking using worn dosimeters. For instance, clinician 407A may wear a dosimeter during a Cath lab procedure during which system 400 also determines a radiation dose via video processing. Following the procedure, system 400 may compare a dose measured by the dosimeter and a dose determined via video processing, and calibrate the video processing algorithm accordingly.

System 400 may perform one or more actions based on the radiation exposure tracking/mapping. As one example, system 400 may generate an exposure report (e.g., a report that indicates a radiation dose experienced by a clinician). As another example, system 400 may separately track radiation exposure doses of multiple body parts of a clinician, and provide exposure report indications for each body part. As another example, system 400 may provide recommendations (e.g., stand in a different location, wear more or less radiation protection equipment, etc.). For instance, system 400 may output a recommendation that a clinician may wear a lighter lead vest (e.g., optimize trade-off between ergonomic risk vs exposure risk; provide more speed, energy, and stamina for some clinicians). Similarly, system 400 may output a recommendation that a clinician wear more protection on their hands.

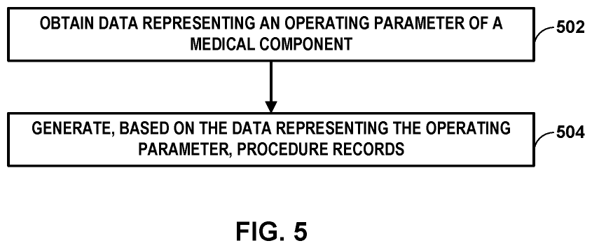

FIG. 5 is a flow diagram illustrating example techniques for tracking and/or monitoring in a Cath lab, in accordance with one or more aspects of the present disclosure. Certain aspects of the example of FIG. 5 are described herein with respect to computing device 200 of FIG. 2 for ease of explanation. It should be noted that the techniques attributed to computing device 200 or components thereof, may be performed by any device of FIG. 1, other devices not shown in FIG. 1 which may be capable of performing such techniques, or any combination thereof.

Processing circuitry 205 may obtain, during performance of procedure in a catheterization lab on a patient, data representing an operating parameter of a medical component (502). The medical component may be a medical device or a pharmacological agent. The operating parameter may be one or more of a location of the medical component, a pressure of the medical component, a size of the medical component, and a serial number of the medical component.

Processing circuitry 205 may obtain the data via one or more input modalities. As one example, processing circuitry 205 may obtain the data via an imaging modality (e.g., video, audio, direct measurement, etc.). For instance, processing circuitry 205 may obtain image data generated by a camera of the catheterization lab; and process the image data to determine the operating parameter of the medical component. In some examples, processing circuitry 205 may process the image data using AI, ML, computer vision, etc. As another example, processing circuitry 205 may receive, via a transmitter attached to the medical component, the data representing the operating parameter of the medical component. The transmitter may include one or more of a radio frequency identification (RFID) transmitter, a near field communication (NFC) transmitter, a radio frequency (RF) transmitter, and a BLUETOOTH transmitter.

Processing circuitry 205 may perform one or more operations based on the obtained data representing the operating parameter. As one example, processing circuitry 205 may generate, based on the data representing the operating parameter, procedure records of the procedure (504). As another example, processing circuitry 205 may include, in the procedure records, video or audio data captured during the procedure. As another example, processing circuitry 205 may include, in the procedure records, data determined from video or audio data captured during the procedure (with or without actually included the video or audio data in the procedure records).

In some examples, processing circuitry 205 may obtain clinical information of the procedure. Processing circuitry 205 may obtain the clinical information via any suitable means. For instance, processing circuitry 205 may obtain, via a display in the catheterization lab (e.g., screen capture), the clinical information of the procedure. Examples of clinical data include, but are not limited to, a fractional flow reserve (FFR), an electrocardiogram (ECG), a heart rate, and a blood pressure. Processing circuitry 205 may, in some examples, include the clinical information in the procedure records.

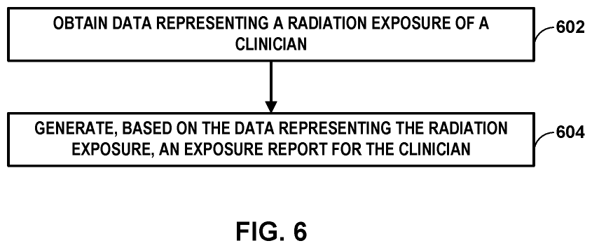

FIG. 6 is a flow diagram illustrating example techniques for tracking and/or monitoring radiation in a Cath lab, in accordance with one or more aspects of the present disclosure. Certain aspects of the example of FIG. 6 are described herein with respect to computing device 200 of FIG. 2 for ease of explanation. It should be noted that the techniques attributed to computing device 200 or components thereof, may be performed by any device of FIG. 1, other devices not shown in FIG. 1 which may be capable of performing such techniques, or any combination thereof.

Processing circuitry 205 may obtain, during performance of procedure in a catheterization lab on a patient, data representing a radiation exposure of a clinician in the catheterization lab (602). Processing circuitry 205 may receive the data representing the radiation exposure from one or both of a dosimeter and/or video processing.

Processing circuitry 205 may perform one or more actions based on the data representing the radiation exposure. As one example, processing circuitry 205 may generate, based on the data representing the radiation exposure, an exposure report for the clinician (604). For instance, processing circuitry 205 may generate a report that indicates how much radiation a clinician was dosed with during the procedure (e.g., in mSv or any other suitable unit). As another example, processing circuitry 205 may cause a display to output a heatmap (e.g., a live heatmap) of the radiation exposure of the clinician.

The techniques described in this disclosure may be implemented, at least in part, in hardware, software, firmware or any combination thereof. For example, various aspects of the described techniques may be implemented within one or more processors or processing circuitry, including one or more microprocessors, digital signal processors (DSPs), application specific integrated circuits (ASICs), field programmable gate arrays (FPGAs), or any other equivalent integrated or discrete logic circuitry, as well as any combinations of such components. The terms “controller”, “processor”, or “processing circuitry” may generally refer to any of the foregoing logic circuitry, alone or in combination with other logic circuitry, or any other equivalent circuitry. A control unit comprising hardware may also perform one or more of the techniques of this disclosure. Such hardware, software, and firmware may be implemented within the same device or within separate devices to support the various operations and functions described in this disclosure. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components.

The techniques described in this disclosure may also be embodied or encoded in a computer-readable medium, such as a computer-readable storage medium, containing instructions. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), or electronically erasable programmable read only memory (EEPROM), or other computer readable media.

This disclosure includes the following non-limiting examples.

Example 1A. A medical system comprising: memory; and processing circuitry communicatively coupled to the memory, the processing circuitry being configured to: obtain, during performance of a procedure in a medical facility on a patient, data representing an operating parameter of a medical component, the medical component comprising a medical device or a pharmacological agent; and generate, based on the data representing the operating parameter, procedure records of the procedure.

Example 2A. The medical system of example 1A, wherein the operating parameter comprises one or more of: a location of the medical component; a pressure of the medical component; a rotational speed of the medical component; power delivery parameters of the medical component; a size of the medical component; and a serial number of the medical component.

Example 3A. The medical system of example 1A or example 2A, wherein, to obtain the data representing the operating parameter of the medical component, the processing circuitry is configured to: obtain image data generated by a camera of the medical facility; and process the image data to determine the operating parameter of the medical component.

Example 4A. The medical system of any of examples 1A-3A, wherein, to obtain the data representing the operating parameter of the medical component, the processing circuitry is configured to: receive, via a transmitter attached to the medical component, the data representing the operating parameter of the medical component.

Example 5A. The medical system of example 4A, wherein the transmitter comprises one or more of a radio frequency identification (RFID) transmitter, a near field communication (NFC) transmitter, a radio frequency (RF) transmitter, or a BLUETOOTH transmitter.

Example 6A. The medical system of example 4A or 5A, wherein the transmitter comprises an active transmitter.

Example 7A. The medical system of example 4A or 5A, wherein the transmitter comprises a passive transmitter.

Example 8A. The medical system of any of examples 4A-7A, further comprising one or more receivers configured to receive the data from the transmitter.

Example 9A. The medical system of example 8A, wherein at least one receiver of the one or more receivers comprises a pad attached to a table on which the patient is placed during the procedure.

Example 10A. The medical system of example 8A, wherein the one or more receivers are configured to wirelessly provide power to the medical component.

Example 11A. The medical system of any of examples 8A-10A, wherein the one or more receivers comprise a plurality of receivers disparately positioned about the medical facility.

Example 12A. The medical system of any of examples 2A-11A, wherein the operating parameter comprises a pressure of a balloon used in performance of the procedure.

Example 13A. The medical system of example 12A, wherein the processing circuitry is configured to: adjust, based on the pressure, operation of an indeflator that inflates and deflates the balloon.

Example 14A. The medical system of any of examples 1A-13A, wherein the processing circuitry is further configured to: obtain, during the performance of the procedure, a representation of audio data from the medical facility.

Example 15A. The medical system of example 14A, wherein, to generate the procedure records, the processing circuitry is further configured to: generate the procedure records based on the representation of the audio data.

Example 16A. The medical system of example 15A, wherein, to generate the procedure records based on the representation of the audio data, the processing circuitry is configured to: generate a transcription of the representation of the audio data; and include, in the procedure records, the transcription.

Example 17A. The medical system of any of examples 14A-16A, wherein the processing circuitry is further configured to: determine a desired operating parameter of the medical component; and output, responsive to determining that the obtained operating parameter is different than the desired operating parameter of the medical component, a warning.

Example 18A. The medical system of example 17A, wherein, to determine the desired operating parameter, the processing circuitry is configured to: process the representation of the audio data to determine the desired operating parameter.

Example 19A. The medical system of any of examples 1A-18A, wherein the processing circuitry is further configured to: obtain, via a display in the medical facility, clinical information of the procedure.

Example 20A. The medical system of example 19A, wherein, to generate the procedure records, the processing circuitry is further configured to: generate the procedure records based on the clinical data.

Example 21A. The medical system of example 19A or 20A, wherein the clinical data comprises one or more of a fractional flow reserve (FFR), an electrocardiogram (ECG), a heart rate, and a blood pressure.

Example 22A. The medical system of any of examples 1A-21A, wherein, to obtain the data representing the operating parameter, the processing circuitry is configured to obtain the data via a medical component storage system from which a clinician obtained the medical component.

Example 23A. The medical system of example 22A, further comprising the medical component storage system.

Example 24A. The medical system of example 22A or 23A, wherein the medical component storage system is configured to output, to the processing circuitry, an indication that the medical component has been removed from the medical component storage system.

Example 25A. The medical system of any of examples 1A-24A, wherein the medical device comprises one or more of: a balloon, a catheter, and a guidewire.

Example 26A. The medical system of any of examples 1A-25A, wherein the pharmacological agent comprises contrast.

Example 1B. A medical system comprising: memory; and processing circuitry communicatively coupled to the memory, the processing circuitry being configured to: obtain, during performance of a procedure in a medical facility on a patient, data representing a radiation exposure of a person in the medical facility; and generate, based on the data representing the radiation exposure, an exposure report for the person.

Example 2B. The medical system of example 1B, wherein, to obtain the data representing the radiation exposure of the person, the processing circuitry is configured to: receive, from a dosimeter worn by the clinician, the data representing the radiation exposure of the person.

Example 3B. The medical system of example 1B or example 2B, wherein the processing circuitry is further configured to: determine one or more parameters of a radiation emitting device positioned in the medical facility, wherein, to obtain the data representing the radiation exposure of the person, the processing circuitry is configured to obtain the data based on the one or more parameters of the radiation emitting device.

Example 4B. The medical system of example 3B, wherein the one or more parameters comprise one or more of a direction of emission, a focus of emission, and an intensity of emission.

Example 5B. The medical system of example 3B or example 4B, wherein, to obtain the data representing the radiation exposure of the person, the processing circuitry is configured to: receive, from one or more cameras positioned in the medical facility, video data; and determine, based on the video data and the one or more parameters of the radiation emitting device, the data representing the radiation exposure of the person.

Example 6B. The medical system of example 5B, wherein the processing circuitry is configured to: determine, based on the video data, a position of the person relative to the radiation emitting device.

Example 7B. The medical system of any of examples 3B-6B, wherein the processing circuitry is configured to: model, based on the one or more parameters of the radiation emitting device, a radiation field; and determine, based on the modeled radiation field, the data representing the radiation exposure of the person.

Example 8B. The medical system of any of examples 1B-7B, wherein, to obtain the data representing the radiation exposure of the person, the processing circuitry is configured to: obtain, for respective body parts of a plurality of body parts of the person, respective data representing radiation exposure of the respective body part.

Example 9B. The medical system of example 8B, wherein the plurality of body parts include one or more of a left hand of the person, a right hand of the person, and a head of the person.

Example 10B. The medical system of example 8B or example 9B, wherein, to generate the exposure report for the person, the processing circuitry is configured to: generate the exposure report to include radiation exposure data for the plurality of body parts of the person.

Example 11B. The medical system of any of examples 1B-10B, wherein: the clinician is a first person of a plurality of persons, the processing circuitry is configured to obtain, for each respective person of the plurality of persons, respective data representing radiation exposure, and the processing circuitry is configured to generate a respective exposure report for each person of the plurality of persons.

Example 12B. The medical system of any of examples 1B-11B, wherein the processing circuitry is further configured to: output, during performance of procedure and based on the data representing the radiation exposure of the person, a heatmap of the radiation exposure of the person.

Example 13B. The medical system of example 12B, wherein, to output the heatmap, the processing circuitry is configured to: output a live heatmap of the radiation exposure of the person.

Example 14B. The medical system of any of examples 1B-13B, wherein the processing circuitry is further configured to: output, based on the data representing the radiation exposure of the clinician, a recommendation for different location for the person to stand.

Example 15B. The medical system of any of examples 1B-14B, wherein the processing circuitry is further configured to: output, based on the data representing the radiation exposure of the person, a recommendation for radiation protection equipment for the person.

Example 16B. Any combination of examples 1A-15B.

Various examples have been described. These and other examples are within the scope of the following claims.

1. A medical system comprising:
memory; and
processing circuitry communicatively coupled to the memory, the processing circuitry being configured to:
obtain, during performance of a procedure in a medical facility on a patient, data representing an operating parameter of a medical component, the medical component comprising a medical device or a pharmacological agent; and
generate, based on the data representing the operating parameter, procedure records of the procedure.

2. The medical system of claim 1, wherein the operating parameter comprises one or more of:
a location of the medical component;
a pressure of the medical component;
a rotational speed of the medical component;
power delivery parameters of the medical component;
a size of the medical component; and
a serial number of the medical component.

3. The medical system of claim 1, wherein, to obtain the data representing the operating parameter of the medical component, the processing circuitry is configured to:
obtain image data generated by a camera of the medical facility; and
process the image data to determine the operating parameter of the medical component.

4. The medical system of claim 1, wherein, to obtain the data representing the operating parameter of the medical component, the processing circuitry is configured to:
receive, via a transmitter attached to the medical component, the data representing the operating parameter of the medical component.

5. The medical system of claim 4, wherein the transmitter comprises one or more of a radio frequency identification (RFID) transmitter, a near field communication (NFC) transmitter, a radio frequency (RF) transmitter, or a BLUETOOTH transmitter.

6. The medical system of claim 4, further comprising one or more receivers configured to receive the data from the transmitter.

7. The medical system of claim 6, wherein at least one receiver of the one or more receivers comprises a pad attached to a table on which the patient is placed during the procedure.

8. The medical system of claim 6, wherein the one or more receivers are configured to wirelessly provide power to the medical component.

9. The medical system of claim 6, wherein the one or more receivers comprise a plurality of receivers disparately positioned about the medical facility.

10. The medical system of claim 2, wherein the operating parameter comprises a pressure of a balloon used in performance of the procedure, and, wherein the processing circuitry is configured to:
adjust, based on the pressure, operation of an indeflator that inflates and deflates the balloon.

11. The medical system of claim 1, wherein the processing circuitry is further configured to:
obtain, during the performance of the procedure, a representation of audio data from the medical facility, wherein, to generate the procedure records, the processing circuitry is further configured to:
generate the procedure records based on the representation of the audio data.

12. The medical system of claim 11, wherein, to generate the procedure records based on the representation of the audio data, the processing circuitry is configured to:
generate a transcription of the representation of the audio data; and
include, in the procedure records, the transcription.

13. The medical system of claim 11, wherein the processing circuitry is further configured to:
determine a desired operating parameter of the medical component; and
output, responsive to determining that the obtained operating parameter is different than the desired operating parameter of the medical component, a warning.

14. The medical system of claim 13, wherein, to determine the desired operating parameter, the processing circuitry is configured to:
process the representation of the audio data to determine the desired operating parameter.

15. The medical system of claim 1, wherein the processing circuitry is further configured to:
obtain, via a display in the medical facility, clinical data of the procedure, wherein, to generate the procedure records, the processing circuitry is further configured to:
generate the procedure records based on the clinical data.

16. The medical system of claim 1, wherein, to obtain the data representing the operating parameter, the processing circuitry is configured to obtain the data via a medical component storage system from which a clinician obtained the medical component.

17. The medical system of claim 16, further comprising the medical component storage system, wherein the medical component storage system is configured to output, to the processing circuitry, an indication that the medical component has been removed from the medical component storage system.

18. A method comprising:
obtaining, during performance of a procedure in a medical facility on a patient, data representing an operating parameter of a medical component, the medical component comprising a medical device or a pharmacological agent; and
generating, based on the data representing the operating parameter, procedure records of the procedure.

19. The method of claim 18, wherein obtaining the data representing the operating parameter of the medical component comprises:
obtaining image data generated by a camera of the medical facility; and
processing the image data to determine the operating parameter of the medical component.

20. Computer-readable storage media storing instructions that, when executed, cause one or more processors to:
obtain, during performance of a procedure in a medical facility on a patient, data representing an operating parameter of a medical component, the medical component comprising a medical device or a pharmacological agent; and
generate, based on the data representing the operating parameter, procedure records of the procedure.
